(19) United States
(12) Patent Application Publication
(10) Pub. No.: US 2011/0027192 A1
(43) Pub. Date: Feb. 3, 2011

(54) SLIM-FIT FAT BURNING WATER

(76) Inventor: Fabio Lanzoni, Tarzana, CA (US)

Correspondence Address: IPC Intellectual Property Connections, INC., 299 Old County Road, Suite 28, San Carlos, CA, 94070, US

(21) Appl. No.: 12/920,707

(22) PCT Filed: Mar. 9, 2009
(86) PCT No.: PCT/US2009/036489

§ 371 Date: Sep. 2, 2010

Related U.S. Application Data
(60) Provisional application No. 61/035,035, filed on Mar. 10, 2008.

(51) Int. Cl. A61K 31/205; A61K 36/00; A61K 31/675; A61K 31/455; A61K 31/714; A61P 3/02; A61P 3/04

(52) U.S. Cl. 424/43; 514/556; 424/777; 514/89; 514/356; 514/52

(57) ABSTRACT

A fat-burning, energy-producing water composition. The water composition contains spring water and a premeasured, effective amount of L-carnitine dissolved in the spring water at a concentration of no less than 100 mg and no more than 2000 mg per 16 oz of spring water. The water composition may further include at least one nutritious component selected from the group consisting of vitamin B-6, L-Lysine. vitamin B-3, vitamin B-12 and any combination thereof. The water composition may be carbonated or uncarbonated, flavored or flavorless. The water composition in bottled water is administered to a mammal for quenching thirst and increasing fat-burning, improving energy, and keeping body slim and fit.

### REFERENCE TO RELATED APPLICATION

This application is a national stage application (under 35 U.S.C. 371) of PCT/US2009/036489 filed Mar. 9, 2009, which claims the priority to U.S. Provisional application Ser. No. 61/035,035 filed Mar. 10, 2008, which is herein incorporated by reference in its entirety.

### FIELD OF THE INVENTION

The present invention relates generally to a fat-burning, energy-producing water composition for increasing the degradation of body fat and providing energy to a subject.

### BACKGROUND OF THE INVENTION

L-Carnitine was first found in muscle extracts in 1905 by Gulewitch and Krimberg. In 1952, Fraenkel isolated L-carnitine from liver extract and gave it the name “vitamin BT.” Fritz in 1958 found that L-carnitine increased the rate at which cells (mitochondria) burned fat. This established L-carnitine's fundamental role in fat oxidation. Lonza patented its unique method for production of L-carnitine in 1983. There was a long interval between discovery and common usage of L-carnitine. For many decades, only a small amount of L-carnitine, extracted from meat, was available for research purposes.

In 1993, an independent expert committee of scientists evaluated the “Generally Recognized As Safe (GRAS)” status of L-carnitine as a dietary supplement. They concluded that L-carnitine and its freely ionizable salts such as L-Carnitine L-Tartrate and L-Carnitine Magnesium Citrate can be safely ingested by adults at 20 mg/kg/day (1200 mg for a 60 kg person) for a broad range of uses. At “Experimental Biology 2001,” Müller reported that oral L-carnitine supplementation improved long chain fatty acid oxidation as measured by ¹³CO₂ exhalation in healthy subjects. In 2004, Wutzke found that oral L-Carnipure® L-Carnitine L-Tartrate supplementation increased long chain fatty acid oxidation in slightly overweight adults. Today, L-carnitine is recognized as a natural, vitamin-like nutrient which plays an important role in human metabolism. It is essential in the utilization of fatty acids and in transporting metabolic energy.

Fatty acids are one of the primary energy sources for the body. β-oxidation is the process by which fatty acids are broken down for the ultimate production of energy. Although fatty acids need to enter the mitochondria for β-oxidation, they are unable to penetrate the inner mitochondrial membrane. L-carnitine is essential to transport long chain fatty acids across the mitochondrial membrane for subsequent fat breakdown and energy production. In addition, L-carnitine has been shown to buffer the bound CoA to free CoA ratio and removes toxic concentrations of acyl-CoA thereby helping to ensure that energy production can continue. Another important function of L-carnitine is the ability to shuttle short chain organic acids from inside the mitochondria to the cytosol. Due to its functional role in energy metabolism, L-carnitine is typically used to support all bodily functions that have a high energy demand.

L-Carnitine is available as a dietary supplement in tablet, capsule, powder, or in a concentrated liquid syrup form. Tablet or capsule forms have to be swallowed typically with a beverage. Many people are averse to swallowing tablets and capsules. In addition, the size of the dosage form would hinder its usage. The powder form would have to be measured each time and mixed with a beverage for consumption. This presented a lack of convenience for many. The concentrated liquid form was flavored and enhanced with sweeteners to modify the taste. Many are opposed to ingesting sugars, artificial colors and flavors, or sweeteners in order to obtain their L-carnitine.

Another constant prior issue stems from the fact that L-carnitine material is very hygroscopic, or attracting moisture. As a result, L-carnitine dry form products would have to be stored in a cool, dry environment; otherwise, the tablets and capsules could fall apart, change color, become sticky, and/or develop an odor.

Moreover, the vast amounts of products on the market today that currently contain L-carnitine provide a very low amount and, as a result, a non-efficacious dosage. Much of the research available on L-carnitine supplementation has been done with 250 mg to 3,000 mg of L-carnitine and occasionally higher. The products that contain minimal dosages of L-carnitine are able to capitalize on the marketing benefits gained on the inclusion of the ingredient, but do not provide much of a fat burning effect. Therefore, the dosage range of L-carnitine supplementation is crucial to the efficacy of the product.

Therefore, a heretofore unaddressed need exists in the art to address the aforementioned deficiencies and inadequacies, especially in connection with the L-carnitine products.

### SUMMARY OF THE INVENTION

In one aspect, the invention relates to a fat-burning, energy-producing water composition. In one embodiment of the invention, the composition includes: (a) spring water; and (b) a premeasured, effective amount of L-carnitine in the spring water at a concentration of no less than 100 mg/16 oz and no more than 2000 mg/16 oz.

In another aspect, the invention relates to a fat-burning, energy-producing water composition, which contains: (a) spring water; and (b) a premeasured, effective amount of L-carnitine in the water at a concentration of no less than 100 mg/16 oz, wherein the water composition contains no or little sweetening additive.

Further in another aspect, the invention relates to a fat-burning, energy-producing water composition, which contains: (a) spring water; (b) a premeasured, effective amount of L-carnitine in the water at a concentration of no less than about 100 mg/16 oz, and no larger than about 2000 mg/16 oz; and (c) at least one nutritious component selected from the group consisting of vitamin B-6, L-Lysine, vitamin B-3, vitamin B-12 and any combination thereof, wherein the composition contains no or little sweetener.

Yet in another aspect, the invention relates to a fat-burning, energy-producing water composition, which consists essentially of: (a) spring water; and (b) a premeasured, effective amount of L-carnitine in the water at a concentration of no less than 100 mg/16 oz.

Further in another aspect, the invention relates to a fat-burning, energy-producing water composition, which consists essentially of (a) spring water; (b) a premeasured, effective amount of L-carnitine in the water at a concentration of no less than about 100 mg/16 oz, and no larger than about 2000 mg/16 oz; and (c) at least one nutritious component selected from the group consisting of vitamin B-6, L-Lysine, vitamin B-3, vitamin B-12 and any combination thereof.

The composition may be either carbonated or uncarbonated. It may be flavorless or may contain a flavoring agent.

In one embodiment of the invention, the water composition contains no or little carbohydrate, fat, sodium, natural or artificial sweeteners, flavoring agents, coloring agents, and/or vegetable glycerin.

In another embodiment of the invention, the water composition further contains fruit juice.

Further in another embodiment of the invention, the water composition further contains at least one nutritious component selected from the group consisting of vitamin B-6, L-Lysine, vitamin B-3, vitamin B-12 and any combination thereof.

In another aspect, the invention relates to a bottled water composition for quenching thirst and keeping slim and fit in a subject comprising any one of the aforementioned water compositions.

Yet in another aspect, the invention relates to a method of keeping slim and fit in a subject, comprising the step of administering the subject one of the aforementioned fat-burning, energy-producing water composition.

These, and other aspects will become apparent from the following description of the preferred embodiment taken in conjunction with the following drawings, although variations and modifications therein may be affected without departing from the spirit and scope of the novel concepts of the disclosure.

The accompanying drawings illustrate one or more embodiments of the invention and, together with the written description, serve to explain the principles of the invention. Wherever possible, the same reference numbers are used throughout the drawings to refer to the same or like elements of an embodiment.

### DETAILED DESCRIPTION OF THE INVENTION

### DEFINITIONS

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

Unless otherwise defined, all technical and scientific terms used herein have the same meaning as commonly understood by one of ordinary skill in the art to which this invention pertains. In the case of conflict, the present document, including definitions will control.

As used herein, “around”, “about” or “approximately” shall generally mean within 20 percent, preferably within 10 percent, and more preferably within 5 percent of a given value or range. Numerical quantities given herein are approximate, meaning that the term “around”, “about” or “approximately” can be inferred if not expressly stated.

As used herein, a “water composition,” shall generally mean a water delivery system, a drink, or a beverage for supplying the body with water and/or for quenching thirst. It may be a transparent, odorless, tasteless liquid, and/or a liquid that looks and taste like water, H₂O. It may contain a flavoring agent if so desired.

As used herein, “spring water” shall generally mean water of a natural resource from which much bottled water comes and is generally imbued with minerals. A “spring” is a point where groundwater flows out from the ground.

As used herein, “tap water” refers to water piped to homes through a tap.

As used herein, “distilled water” or “deionized water” refers to “purified water,” which is water from any source that is physically processed to remove impurities.

As used herein, “carbonated water,” also known as sparkling water, fizzy water and seltzer, is water into which carbon dioxide gas has been dissolved.

As used herein, a “sweetener” is a food additive which adds the basic taste of sweetness to a food, e.g., sugar; artificial sweeteners are sugar substitutes. A “sweetener” is any of various natural and artificial substances that provide a sweet taste in food and beverages.

As used herein, sugar includes sucrose, lactose, and fructose.

As used herein, a “flavoring agent” or “flavorant” is defined as a substance that gives another substance flavor, altering the characteristics of the solute, causing it to become sweet, sour, tangy, etc.

As used herein, “fruit juice” refers to a liquid naturally contained in fruit. Juices include but are not limited to apple, orange, grapefruit, pineapple, tomato, passion fruit, mango, carrot, grape, cranberry, lemon, tangerine, strawberry, kiwi, lime, and pomegranate. It may include a combination of a variety of fruits into a single juice.

As used herein, “bottled water composition” is drinking water packaged in bottles for individual consumption and retail sale.

### EXAMPLES

Without intent to limit the scope of the invention, exemplary instruments, apparatus, methods and their related results according to the embodiments of the present invention are given below. Note that titles or subtitles may be used in the examples for convenience of a reader, which in no way should limit the scope of the invention. Moreover, certain theories are proposed and disclosed herein; however, in no way they, whether they are right or wrong, should limit the scope of the invention so long as the invention is practiced according to the invention without regard for any particular theory or scheme of action.

### EXAMPLE 1

A pre-measured amount of L-carnitine was added into water at a final concentration range from about 100 mg to about 2000 mg per 16 fluid ounces (oz) of water. The pre-measured amount of L-carnitine was completely soluble in the water. The hygroscopic nature of L-carnitine is no longer a factor with the present invention since it's already dissolved in the water in a container, such as a bottle.

| | |
|---|---|
| L-Carnitine | about 100-2000 mg |
| Spring water | about 16 oz |

The primary ingredient, L-carnitine, has been researched for over 40 years and been found to be essential for energy production and fat metabolism. Fatty acid oxidation is the process by which fatty acids are broken down in the cell mitochondria for the ultimate production of energy. Although fatty acids need to enter the mitochondria for oxidation, they are unable to penetrate the inner mitochondrial membrane. L-carnitine is the essential component to transport long chain fatty acids across the mitochondrial membrane for subsequent fat breakdown and energy production.

It has been identified that a daily dosage of about 2000 mg is required for L-carnitine to be effective. With water being utilized as a delivery system, the difficulties in swallowing capsules and tablets could be alleviated, as were the hindrances of powders and flavored syrups. In one embodiment of the invention, the L-carnitine dosage in the water composition is selected within a dosage range that does not alter the taste or odor present in water. In another embodiment of the invention, the resulting water and/or bottled water composition contains no or little flavors, colors, sweeteners, carbohydrates, fat, calories, sodium and/or vegetable glycerin. The amount of L-carnitine per 16 oz ranges from about 100 to about 2000 mg. For example, the amount of L-carnitine per 16 oz is no less than 100 mg, or no less than 200 mg, 300 mg, 400 mg, 500 mg, or 600 mg, and is no more than 700 mg, 800 mg, 900 mg, 1000 mg, 1100 mg, 1200 mg, 1300 mg, 1400 mg, 1500 mg, 1600 mg, 1700 mg, 1800 mg, 1900 mg, or 2000 mg.

### EXAMPLE 2

The following ingredients were added for an 8 oz per serving basis:

| | | |
|---|---|---|
| L-Carnitine | 250 | mg |
| Vitamin B-6 (Pyridoxine HCL) | 1 | mg |
| L-Lysine | 1 | mg |
| Vitamin B-3 (Niacinamide) | 0.5 | mg |
| Vitamin B-12 (Cyanocobalamin) | 3 | μg |
| Spring water | 8 | oz |

### EXAMPLE 3

The product is a 16 oz (about 500 ml) ready to drink bottled water containing:

| | | |
|---|---|---|
| L-Carnitine | 500 | mg |
| Vitamin B-6 (Pyridoxine HCL) | 2 | mg |
| L-Lysine | 2 | mg |
| Vitamin B-3 (Niacinamide) | 1 | mg |
| Vitamin B-12 (Cyanocobalamin) | 6 | μg |
| Spring water | 16 | oz |

The recommended amount to drink will be 4 bottles per day, yielding a total of 64 oz of fluid and 2000 mg L-carnitine. This fat-burning blend of vitamins and nutrients has been scientifically researched and designed for the addition into water with complete solubility and efficacy. The water's taste, texture, physical properties and characteristics are not adversely affected, if anything the taste is enhanced. With water as the delivery system, the fat-burning, energy-producing water and/or bottled water composition of the invention is a highly convenient method for consumers to burn fat while using a medium that they are already accustomed to.

The invention presents tremendous, economic potential and commercial applications. There is a dramatic increase in the number of overweight individuals, both domestically and internationally. This is looked upon as a precursor to obesity, heart disease, diabetes, and a variety of serious health conditions. Proper diet, exercise, and methods to increase fat burning have become crucial for our health and longevity. The enormous economic potential and commercial application of a fat-burning method utilizing a medium has already gained worldwide acceptance. Millions of people worldwide drink bottled water daily as their primary beverage of choice. The fat-burning, energy production of the invention is the perfect substitute for those individuals seeking to burn fat, increase their energy levels, and build endurance while drinking water that they're already accustomed to drinking. The fact that the water has not been altered in terms of taste, color, flavor, or texture while providing significant fat-burning potential in each bottle distinguishes the fat-burning, energy-producing water of the invention as an enhanced water with a significant functional and direct physiological effect. The water composition may optionally further contain flavoring agents and/or fruit juice if desired.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments and examples were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the present invention pertains without departing from its spirit and scope. Accordingly, the scope of the present invention is defined by the appended claims rather than the foregoing description and the exemplary embodiments described therein.

Some references, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference was individually incorporated by reference.

1-25. (canceled)

26. A fat-burning, energy-producing water composition comprising:
a) spring water, and
b) a premeasured, effective amount of L-carnitine in the spring water at a concentration of no less than 100 mg/16 oz and no more than 2000 mg /16 oz.

27. The water composition of claim 26, wherein the composition contains little or no carbohydrate, fat, sodium, natural or artificial sweeteners, flavoring agents, or coloring agents.

28. The water composition of claim 26, wherein the composition is either carbonated or uncarbonated.

29. The water composition of claim 26, wherein the water composition is flavorless, or further contains a flavoring agent.

30. The water composition of claim 26, further comprising fruit juice.

31. The water composition of claim 26, further comprising at least one nutritious component selected from vitamin B-6, L-Lysine, vitamin B-3, and vitamin B-12.

32. The water composition of claim 31, wherein the composition is either carbonated or uncarbonated.

33. A bottled water composition for quenching thirst and keeping slim and fit in a subject comprising the water composition of claim 31.

34. A bottled water composition for quenching thirst and keeping slim and fit in a subject comprising the water composition of claim 26.

35. A fat-burning, energy-producing water composition comprising:
a) spring water; and
b) a premeasured, effective amount of L-carnitine in the water at a concentration of no less than 100 mg/16 oz,
wherein the water composition contains no or little sweetening additive.

36. The water composition of claim 35, wherein the composition is either carbonated or uncarbonated.

37. The water composition of claim 35, wherein the water composition is flavorless, or further contains a flavoring agent.

38. The water composition of claim 35, further comprising at least one nutritious component selected from vitamin B-6, L-Lysine, vitamin B-3, and vitamin B-12.

39. The water composition of claim 38, wherein the composition is either carbonated or uncarbonated.

40. The water composition of claim 38, wherein the water composition is flavorless, or further comprises a flavoring agent.

41. A fat-burning, energy-producing water composition comprising:
(a) water, and
(b) a premeasured, effective amount of L-carnitine in the water at a concentration of no less than about 100 mg/16 oz, and no larger than about 2000 mg/16 oz;
wherein the composition contains little or no sweetener.

42. The fat-burning, energy-producing water composition of claim 41, further comprising:
at least one nutritious component chosen from vitamin B-6, Lysine, vitamin B-3, and vitamin B-12.

43. The fat-burning, energy-producing water composition of claim 42, wherein the water is spring water and the composition is either carbonated or uncarbonated.

44. The water composition of claim 42, wherein the water composition is either flavorless, or further comprises a flavoring agent.

45. The water composition of claim 42, wherein the composition contains little or no carbohydrate, fat, sodium, flavoring agents, coloring agents or vegetable glycerin.
